(19) United States
(12) Patent Application Publication
(10) Pub. No.: US 2023/0162836 A1
(43) Pub. Date: May 25, 2023

(54) DATA DUCTS FOR PROCESSING OF MEDICAL DATA

(71) Applicant: GE PRECISION HEALTHCARE LLC, Wauwatosa, WI (US)

(72) Inventors: Julien Stegle, Strasbourg (FR); Adam Raba, Schiltigheim (FR); Céline Caldini-Queiros, Schiltigheim (FR)

(21) Appl. No.: 17/702,410

(22) Filed: Mar. 23, 2022

Related U.S. Application Data
(60) Provisional application No. 63/281,798, filed on Nov. 22, 2021.

(51) Int. Cl. G16H 30/20

(52) U.S. Cl. CPC G16H 30/20

(57) ABSTRACT

Systems and methods for data ducts for use in data processing pipelines for processing medical data (e.g., DICOM data) are disclosed. Compared to current streaming APIs, the disclosed ducts enable data processing logic validation, data lineage in terms of end-to-end transformation, data processing audit logs, and error control management. The disclosed ducts provide a higher-level encapsulation of processing APIs and force contractual exchanges, which allows for logical validation and control of each processing step or each group of processing steps. By enforcing contractual exchanges, the ducts (as well as a larger data processing pipeline that includes these ducts) can be logically validated, both in terms of provided data and how this data is processed. More specifically, the disclosed ducts can ensure that various data processing pipelines are properly fed with the proper data and, if an error occurs, can track the error and easily assess its impact.

### CROSS-REFERENCE

This application claims priority from and the benefit of U.S. Provisional Patent Application No. 63/281,798, entitled “DATA DUCTS FOR PROCESSING OF MEDICAL DATA,” filed Nov. 22, 2021, which is herein incorporated by reference in its entirety for all purposes.

### BACKGROUND

The subject matter disclosed herein generally relates to processing medical data, and, more specifically, relates to improving validation, error handling, and traceability in the processing of medical data.

Streaming application programming interfaces (APIs) are commonly used to facilitate the transfer and processing of medical data. In general, streaming APIs are focused on independent processing units, parallel scaling, and directed acyclic graphs (DAGs). However, streaming APIs lack logical data processing checks, data traceability and processing audit capabilities, or error recovery based on where the error occurred in the process. As such, while steaming APIs are generally considered easy to use for new developers and offer business-oriented processing units, steaming APIs are generally unable to provide error recovery mechanisms, contractual-based exchanges, or group management of a set of transformations.

Currently existing data processing pipelines, often based on streaming APIs and/or directed acyclic diagrams, lack contracts in terms of data exchange and do not offer the possibility to group processing units. As a result, for existing data processing pipelines, it is not possible to properly track data, determine how the data is processed, control lifecycles, or provide error management. The lack of tracking also makes code prone to human error, which makes some errors difficult or impossible to detect before the pipeline is deployed in a production environment.

### BRIEF DESCRIPTION

With the foregoing in mind, present embodiments are directed to systems and methods for to store Digital Imaging and Communications in Medicine (DICOM) data ducts (also referred to herein as simply “ducts”) for use in the processing of medical data. DICOM is a standard for the communication and management of medical imaging information and related data. The disclosed ducts are generally motivated by the lack of high-level streaming APIs in relevant development languages (e.g., Python), by the need for error management in processing, by the need for supporting or implementing processing audits, and/or by the need for data traceability. For example, compared to current streaming APIs, the disclosed ducts enable data processing logic validation, data lineage in terms of end-to-end transformation, data processing audit logs, and error control management.

The disclosed ducts provide a higher-level encapsulation of processing APIs and force contractual exchanges, which allows for logical validation and control of each processing step or each group of processing steps. By enforcing contractual exchanges, the ducts (as well as a larger data processing pipeline that includes these ducts) can be logically validated, both in terms of provided data and how this data is processed, which is important to businesses dealing with private information. More specifically, the disclosed ducts can ensure that various data processing pipelines are properly fed with the suitable data and, if an error occurs, can track the error and easily assess its impact. It may be appreciated that this enables these ducts to provide automated data lineage generation. Additionally, the disclosed ducts support error policies, which allows for routing depending on when and where the error occurs in the duct or the data processing pipeline.

In an embodiment, a computing system includes at least one memory configured to store a database and instructions of a data processing pipeline for processing DICOM data related to a study and at least one processor configured to execute the stored instructions of the data processing pipeline to perform actions. The actions include ingesting, via a plurality of ingestion ducts of the data processing pipeline, a plurality of DICOM files of the study by: parsing each of the plurality of DICOM files to populate a corresponding plurality of dictionaries, storing the data of the plurality of dictionaries in the database, updating a shared context of the data processing pipeline with identifiers that reference the stored data of each of the plurality of dictionaries within the database, and providing the plurality of dictionaries as input to an accumulation duct of the data processing pipeline. The actions also include accumulating, via the accumulation duct, the plurality of dictionaries received from the plurality of ingestion ducts and the identifiers of the shared context to populate a registry, and in response to determining, based on the registry, that each of the plurality of DICOM files of the study has been ingested, providing the registry as input to an enhancement duct of the data processing pipeline. The actions further include enhancing, via the enhancement duct, the stored data of the plurality of dictionaries within the database for the study, which is accessed within the database using the identifiers of the registry received from the accumulation duct.

In an embodiment, a computer-implemented method of operating a data processing pipeline includes ingesting, via a plurality of ingestion ducts of the data processing pipeline, a plurality of DICOM files of a study by: parsing each of the plurality of DICOM files to populate a corresponding plurality of dictionaries, storing the data of the plurality of dictionaries in a database, updating a shared context of the data processing pipeline with identifiers that reference the stored data of each of the plurality of dictionaries within the database, and providing the plurality of dictionaries as input to an accumulation duct of the data processing pipeline. The method also includes accumulating, via the accumulation duct, the plurality of dictionaries received from the plurality of ingestion ducts and the identifiers of the shared context to populate a registry, and in response to determining, based on the registry, that each of the plurality of DICOM files of the study has been ingested, providing the registry as input to an enhancement duct of the data processing pipeline. The method further includes enhancing, via the enhancement duct, the stored data of the plurality of dictionaries within the database for the study, which is accessed within the database using the identifiers of the registry received from the accumulation duct.

In an embodiment, a non-transitory, computer-readable medium stores instructions of a data processing pipeline executable by a processor of a computing system. The instructions include instructions to ingest, via a plurality of ingestion ducts of the data processing pipeline, a plurality of DICOM files of the study by: parsing each of the plurality of DICOM files to populate a corresponding plurality of dictionaries, storing the data of the plurality of dictionaries in a database, updating a shared context of the data processing pipeline with identifiers that reference the stored data of each of the plurality of dictionaries within the database, and providing the plurality of dictionaries as input to an accumulation duct of the data processing pipeline. The instructions also include instructions to accumulate, via the accumulation duct, the plurality of dictionaries received from the plurality of ingestion ducts and the identifiers of the shared context to populate a registry, and in response to determining, based on the registry, that each of the plurality of DICOM files of the study has been ingested, providing the registry as input to an enhancement duct of the data processing pipeline. The instructions further include instructions to enhance, via the enhancement duct, the stored data of the plurality of dictionaries within the database for the study, which is accessed within the database using the identifiers of the registry received from the accumulation duct.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagram of a Digital Imaging and Communications in Medicine (DICOM) data duct implemented as part of a DICOM data processing pipeline, in accordance with embodiments of the present technique;

FIG. 2 is a diagram illustrating DICOM acquisition for an embodiment of a DICOM message duct of a DICOM data processing pipeline, in accordance with embodiments of the present technique;

FIG. 3 is a diagram illustrating DICOM parsing for the embodiment of the DICOM message duct, in accordance with embodiments of the present technique;

FIG. 4 is a diagram illustrating persistence output for the embodiment of the DICOM message duct, in accordance with embodiments of the present technique;

FIG. 5 is a diagram illustrating a first accumulator output for the embodiment of the DICOM message duct, in accordance with embodiments of the present technique;

FIG. 6 is a diagram illustrating a second accumulator output for the embodiment of the DICOM message duct, in accordance with embodiments of the present technique;

FIG. 7 is a diagram illustrating association acquisition for an embodiment of a DICOM association duct of a DICOM data processing pipeline, in accordance with embodiments of the present technique;

FIG. 8 is a diagram illustrating DICOM association file parsing for the embodiment of the DICOM association duct, in accordance with embodiments of the present technique;

FIG. 9 is a diagram illustrating persistence output for the embodiment of the DICOM association duct, in accordance with embodiments of the present technique;

FIG. 10 is a diagram illustrating accumulator output for the embodiment of the DICOM association duct, in accordance with embodiments of the present technique;

FIG. 11 is a diagram illustrating registry data acquisition for an embodiment of a DICOM enhancement duct of a DICOM data processing pipeline, in accordance with embodiments of the present technique;

FIG. 12 is a diagram illustrating persistence output for the embodiment of the DICOM enhancement duct, in accordance with embodiments of the present technique;

FIG. 13 is a diagram illustrating enhancement calculations for the embodiment of the DICOM enhancement duct, in accordance with embodiments of the present technique;

FIGS. 14, 15, 16, and 17 illustrate example communications between the components of various embodiments of DICOM data ducts, as well as other components of the system, in accordance with embodiments of the present technique;

FIG. 18 is a diagram illustrating a data interpretation stage and a data manipulation stage for an embodiment of a DICOM message duct, in accordance with embodiments of the present technique;

FIG. 19 is a diagram illustrating how multiple database handlers cooperate to store data within a database for an example embodiment of a DICOM message duct, in accordance with embodiments of the present technique; and

FIG. 20 is a diagram illustrating an example embodiment of a DICOM data processing pipeline that includes a plurality of DICOM data ducts, in accordance with embodiments of the present technique.

### DETAILED DESCRIPTION

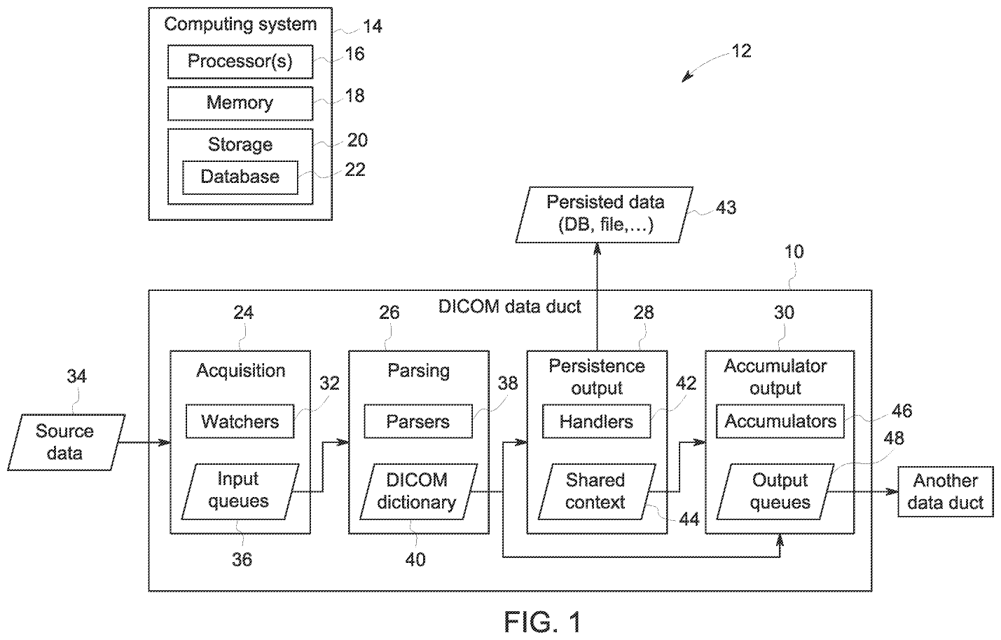

FIG. 1 is a diagram of a DICOM data duct 10, which may be implemented as part of a DICOM data processing pipeline 12, in accordance with embodiments of the present technique. The DICOM data duct 10 and/or the DICOM data processing pipeline 12 may be implemented using at least one computing system 14 having one or more electronic processors 16, at least one memory 18 (e.g., random access memory (RAM), read-only memory (ROM), and at least one electronic storage 20 (e.g., a hard disk device, a solid state disk device) that hosts a suitable file system. In certain embodiments, the storage 20 includes at least one database 22 having one or more database tables configured to store Digital Imaging and Communications in Medicine (DICOM) data. DICOM is a standard for the communication and management of medical imaging information and related data. As discussed below, the computing system 14 includes specialized instructions in the form of watchers, parsers, handlers, accumulators, and so forth, which, when performed by the one or more processors 16 of the computing system 14, result in a specialized computing system for processing DICOM data. Moreover, as mentioned above and discussed below, embodiments of the DICOM data duct 10 and DICOM data processing pipeline 12 disclosed herein improve data processing logic validation, data lineage tracking, data processing audit logs, and error control management when processing DICOM data.

The illustrated DICOM data duct 10 (also referred to herein as “data duct” or simply “duct”) includes a number of subsystems or stages that cooperate to suitably intake, process, and store DICOM data. For the embodiment illustrated in FIG. 1, the stages of the DICOM data duct 10 include an acquisition stage 24, a parsing stage 26, a persistence output stage 28, and an accumulator output stage 30. As discussed below, FIG. 1 represents a generalized DICOM data duct 10, which may be more specifically implemented as a DICOM message duct, a DICOM association duct, a DICOM secondary capture duct, a DICOM accumulation duct, or a DICOM enhancement duct, as discussed below. It may be appreciated that, in other embodiments, the DICOM data duct 10 may have additional or fewer stages, and the stages may be grouped or arranged in different manners, in accordance with the present disclosure. Additionally, the DICOM data processing pipeline 12 may include any suitable number of interconnected DICOM data ducts 10, as discussed below.

For the embodiment illustrated in FIG. 1, the acquisition stage 24 of the DICOM data duct 10 includes one or more watchers 32 (e.g., file system watchers, memory location watchers, database watchers) that monitor a particular location (e.g., a file system location, a memory location, a database location) of the computing system 14 to determine whether new DICOM source data 34 is available to be processed by the duct 10. The watchers 32 may then load a reference to the DICOM source data 34 detected in the monitored location into one or more input queues 36 of the duct 10 for processing. For example, in certain embodiments, the one or more input queues 36 may be populated with a file uniform resource identifier (URI), a file stream, a dictionary (e.g., a Python dictionary), an object (e.g., a Python object), or another suitable source.

For the embodiment illustrated in FIG. 1, the parsing stage 26 of the DICOM data duct 10 includes one or more parsers 38, each having a respective processing engine or a set of processing steps to be performed on the DICOM source data 34 indicated by the input queues 36 to populate a DICOM dictionary 40 (e.g., a Python dictionary storing DICOM data). In certain embodiments, multiple parsers 38 may be chained to split different processing domains. For example, a collection of chained parsers 38 may include: a first parser that opens a DICOM file based on a file URI in the input queues 36 and reads the contents, a second parser that maps DICOM tag content to the DICOM dictionary 40, and a third parser that deciphers a particular private DICOM tag.

For the embodiment illustrated in FIG. 1, the persistence output stage 28 of the DICOM data duct 10 includes one or more handlers 42 (e.g., database handlers, file handlers) that ensure that at least a portion of the data of the populated DICOM dictionary 40 is suitably persisted (e.g., stored within the database 22 or suitable files in storage 20) for later access. In certain embodiments, the persistence output stage 28 uses the one or more handlers 42 of the duct 10 to store processed data 43 within the database 22, while ensuring that a shared context 44 of the duct 10 or pipeline 12 is updated to include identifiers for the stored data within the database 22. For example, the shared context 44 may be a memory space that is accessible to the components of the DICOM data duct 10 or the DICOM data processing pipeline 12 to enable these components to share particular information (e.g., calculated values, identifiers).

For the embodiment illustrated in FIG. 1, the accumulator output stage 30 of the DICOM data duct 10 includes one or more accumulators 46 that accumulate at least some of the data of the DICOM dictionary 40 and/or the shared context 44 to be provided to another DICOM data duct 10 of the DICOM data processing pipeline 12 in one or more output queues 48. In certain embodiments, like the parsers 38 of the duct 10, the outputs of the persistence output stage 28 and/or the accumulator output stage 30 can be chained. For example, a collection of chained outputs of the DICOM data duct 10 may include: a first persistence output to store image level data in the database 22, a second accumulation output to update image level references in the shared context 44, a third persistence output to store series level data in the database 22, a fourth accumulation output to update series level references in the shared context 44, and a fifth persistence output to store link between images and series in the database 22. It may be appreciated that chaining outputs without explicit links enables certain outputs to operate independently from each other, certain outputs to consume each other (e.g., reducing database querying to retrieve previously created objects IDs, and so forth), and certain independent outputs to crash without affecting each other, such that data processing continues. In certain embodiments, such as when the DICOM data duct 10 represents a DICOM enhancement duct, the accumulator output stage 30 may additionally or alternatively include applying enhancement calculations, as discussed below.

In certain embodiments, at least a portion of the output queues 48 of the DICOM data duct 10 are associated with or directly serve as input queues of other ducts and/or other portion of the DICOM data processing pipeline 12. In certain embodiments, the output queues 48 can also serve as a waiting point or staging area for all data to be gathered before beginning further processing (e.g., within a new duct). In certain implementations, an accumulator 46 and output queue 48 can be shared between two or more ducts, and may be implemented as a DICOM accumulation duct, as discussed below. In an example, the outputs of the output queues 48 may include a first output to store references of processed images from a DICOM message duct, a second output to store references of processed association from a DICOM association duct, and a third output that generates an input (e.g. a signal to begin processing) when both references above are matching (e.g., part of a common study). For this example, the first and second outputs may not be chained, but can be performed in an asynchronous manner.

Below is an example involving an embodiment of a DICOM data processing pipeline 12 that includes an embodiment of a DICOM message duct 10A, an embodiment of a DICOM association duct 10B, and an embodiment of a DICOM enhancement duct 10C. As discussed below, in embodiments of the DICOM data processing pipeline 12, these ducts cooperate to process, persist, and enhance received DICOM data. Each of these ducts is separately discussed and described below. For this example, the DICOM data being processed by the pipeline consists of two DICOM files with respective ultrasound images received in one association and a corresponding DICOM association file. More specifically, for this example, the two ultrasound images are received with a Study Instance Unique Identifier (UID): “1.2.3.4”, the first image having a Service-Object Pair (SOP) Instance UID of “SOP1”, and the second having a SOP Instance UID of “SOP2”.

### DICOM Message Duct

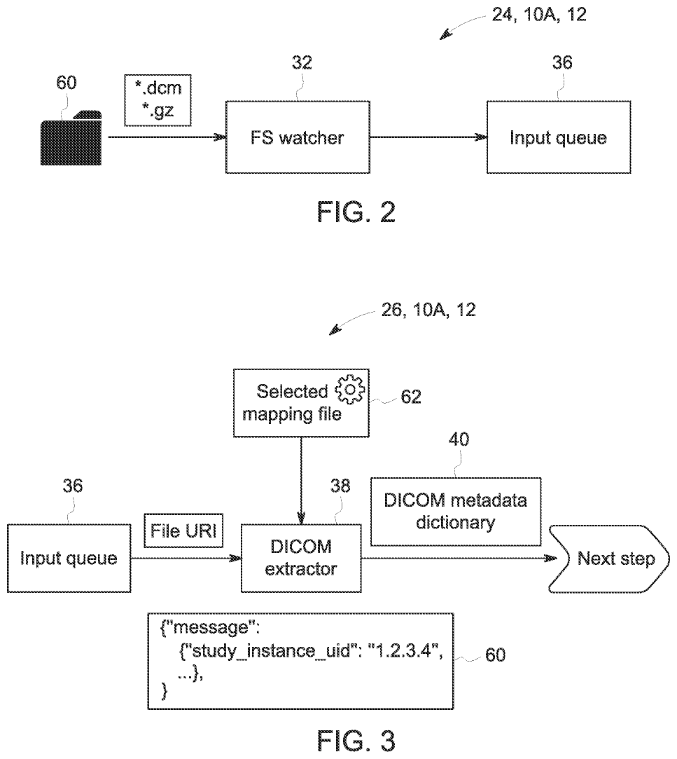

FIGS. 2-6 are diagrams illustrating portions of an example embodiment of a DICOM message duct 10A, which is an example of an ingestion duct of the DICOM data processing pipeline 12. The purpose of the DICOM message duct 10A is generally to parse and store message and/or image data from DICOM files present in a particular location of the file system. As such, FIGS. 2-6 illustrate how objects are processed during the DICOM data ingestion.

FIG. 2 illustrates the acquisition stage 24 of the example DICOM message duct 10A. Each time a suitable file 60 (e.g., a file having a .dcm or .gz extension) is written to a predetermined location of the file system in the storage 20 of the computing system 14, a watcher 32 of the duct 10A (e.g., a listener or file system watcher based on a Python watchdog library) adds the file uniform resource identifier (URI) into an input queue 36 of the duct 10A, which may be implemented as a Python queue in certain embodiments. Once the file 60 has been added to the input queue 36, it becomes available for parsing, as discussed below.

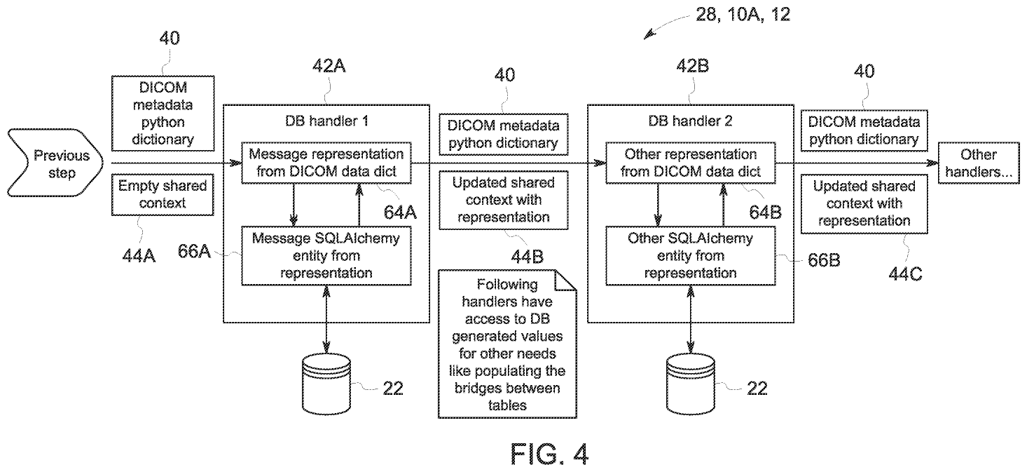

For the example embodiment of the DICOM message duct 10A, FIG. 3 illustrates the parsing stage 26. The parsing stage 26 involves the parsing of DICOM files 60 (e.g., DICOM source data 34) that have been added to the input queue 36. Once a file 60 is available in the input queue 36, it is opened and parsed by at least one DICOM extractor component 38 (e.g., a DICOM parser) of the duct 10A using a mapping JavaScript Object Notation (JSON) file 62 to populate at least one DICOM dictionary 40 (e.g., a DICOM metadata dictionary) in memory 18. For certain embodiments implemented in Python, the DICOM file 60 indicated by the file URI may be opened using the pydicom library. One benefit to using the mapping JSON file 62 is that, since the mapping JSON file 62 is separate from the software instructions of the duct 10A, and since the JSON mapping file 62 is generally configured based on business-specific considerations, this advantageously enables certain users (e.g., business teams) to work independently from developers when defining this mapping.

For instance, the following is a partial example of a mapping JSON file 62 for an embodiment of the DICOM message duct:

{
“message.study_instance_uid”: {
“type”: “uid”,
“tags”: [
[
“0020”,
“000d”
]
],
“operation”: “None”
},
...
}

As a result of the mapping in this example, a DICOM dictionary 40 (e.g., a Python nested DICOM dictionary) may be created in memory with the following value:

data[“message”][“study_instance_uid”]=<value from tag (0020,000d)>

As such, at the conclusion of the parsing stage 26, the DICOM message duct 10A includes at least one DICOM dictionary 40 in memory 18 that is populated with information extracted from the DICOM file 60 and that is available for further processing by other components of the duct 10A. In certain embodiments, multiple sub-dictionaries can be used. For example, a “message.study_instance_uid” and “study.study_datetime” mapping will create a dictionary with keys “message” and “study”. In certain embodiments, additional operations can be applied based on a configuration of the parsers 38 of the parsing stage 26. For example, the parsers 38 may be configured to group or combine the values of two different DICOM tags (e.g., a date tag and a time tag).

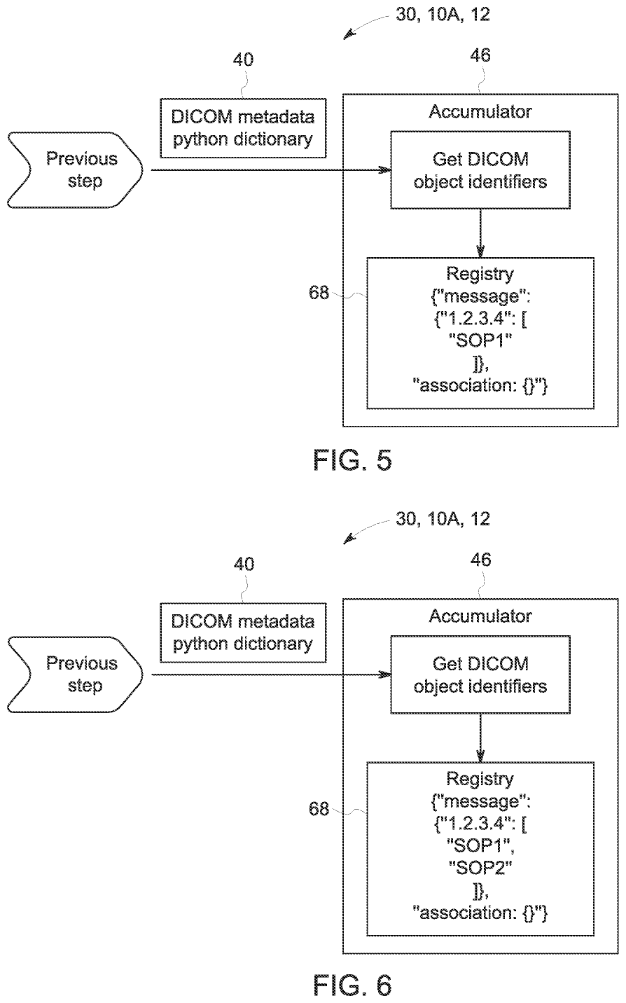

For the example embodiment of the DICOM message duct 10A, FIG. 4 illustrates the persistence output stage 28. For the illustrated example, once the one or more DICOM dictionaries 40 have been populated, they are provided to one or more handlers 42 of the persistence output stage 28, which are illustrated as DB handler 42A and DB handler 42B in FIG. 4. For the illustrated embodiment, the persistence output stage 28 uses database (DB) subunits, referred to as DB handlers, dedicated to the persistence of a single entity (e.g., a message), which allows code to be split among multiple classes. It may be appreciated that this reduces “big blob” effects and enables each persistence output to have customizable actions that are specifically tailored to particular business needs, as well as multiple event levels for customer data auditing. In certain embodiments, these handlers 42 use dedicated mapping code, allowing the auto-generation of data lineage.

For the example embodiment of the DICOM message duct 10A, before the data from the DICOM dictionaries 40 is written to the database 22 in the persistence output stage 28, intermediate objects 64A and 64B are first created in memory 18, referred to herein as “representations”. After creation, each of the representations 64A and 64B is persisted (e.g., stored within the database) using a respective, suitable database object 66A and 66B (e.g., a SQLAlchemy entity), and then the shared context 44 is subsequently updated with suitable identifiers (e.g., database-generated identifiers, UIDs, SOP UIDs, key values, DICOM object identifiers) that reference the persisted data within the database 22. For example, at the beginning of the persistence output stage 28, the shared context 44A may initially be empty, and may be updated with suitable identifiers determined by the DB handler 42A during the first persistence output (as indicated by the shared context 44B), and may be again updated with suitable identifiers determined by the DB handler 42B during the second persistence output (as indicated by the shared context 44C). In certain embodiments, after the shared context 44 has been updated to include these identifiers, other components (e.g., other handlers) of the duct 10A (or the pipeline 12) may have access to these identifiers throughout processing of the DICOM data. In certain embodiments, the DICOM message data may be stored in a message_origin table, or another suitable table of the database 22. It may be appreciated that using representations 64 in this manner avoids having database objects (e.g., SQLAlchemy objects 66) flowing through the DICOM message duct 10A, since each of these objects is tied to an active session. In certain embodiments, before persisting the contents of the DICOM dictionary 40 to the database 22, the DICOM dictionary 40 is validated to ensure that the handlers 42 are executed in a suitable order.

For instance, the following is a partial example of a representation 64 (e.g. a representation object) for a message:

class Message(Representation):
uid: int = None
creation_datetime: datetime = None
study_instance_uid: Optional [str]
...

The following is a partial example of an SQLAlchemy entity 66 configured to persist the example message representation above:

```
class Message(DataBase):
tablename_ = ‘message’
  uid = Column(BigInteger( ), primary_key=True, comment=‘a Dicom
  message’)
creation_datetime = Column(DateTime, nullable=False, index=True,
server_default=text(“current_timestamp( )”))
  study_instance_uid = Column(String(75, ‘utf8_bin’))
  ...
```

Also, in certain embodiments, each of the handlers 42 of the persistence output stage 28 may define what information from the shared context 44 of the DICOM message duct 10A should be accessible in order for each handler to be able to store the appropriate data in the database 22. In certain embodiments, this may be implemented using one or more bridge tables and one or more bridge table handlers. For instance, the following is an example of a message handler class, as well as a bridge message image handler class that handles associations between messages and images:

# Message handler
class MessageHandler(Handler):
_requires_ = ( )
_provides_ = ((‘message’, _representations.Message),
(‘messsage.origin’, _representations.MessageOrigin))
...
# Bridge handler between messages and images
class BridgeMessageImageHandler(Handler):
_requires_ = ((‘image’, _representations.Image), (‘message’,
_representations.Message))
_provides_ = ( )
...

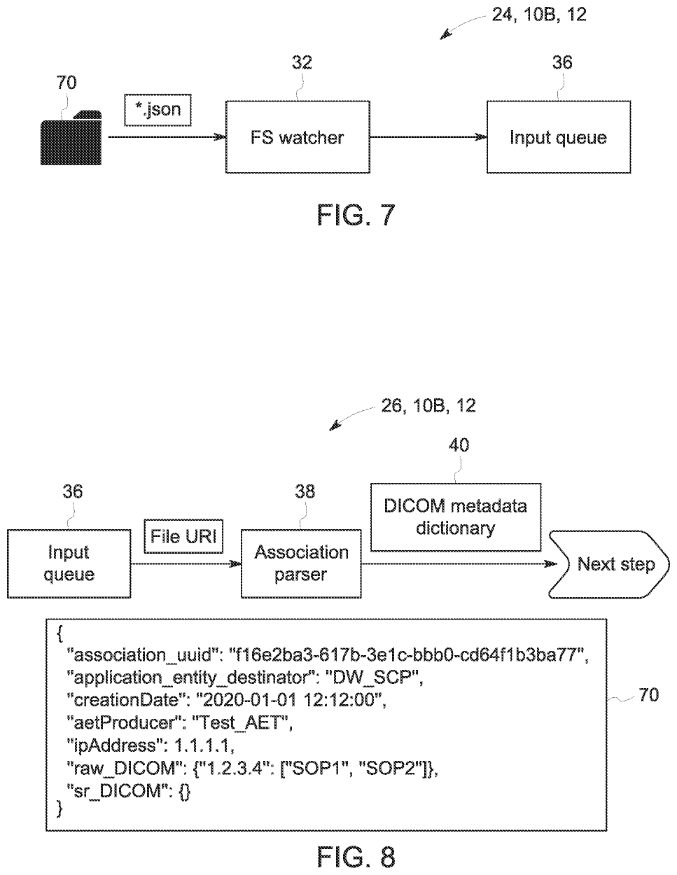

For the example embodiment of the DICOM message duct 10A, FIG. 5 illustrates the accumulator output stage 30. After the DICOM data is stored in the database 22 in the persistence output stage 28, the DICOM dictionary 40 is subsequently provided to the accumulator (also referred to herein as “an association-processed accumulator”), which may be part of the DICOM message duct 10A, or may be part of a DICOM accumulation duct or a DICOM enhancement duct in certain embodiments, as discussed below. The accumulator 46 receives the DICOM dictionary 40, and accesses the identifiers (e.g., DICOM object identifiers) from the updated shared context 44, to create a registry 68 in memory 18. This registry 68 is a data structure that generally stores identifiers (e.g., identifying information, database-generated identifiers, UIDs, SOP UIDs, key values, associations) regarding the DICOM data that has been received, processed, and persisted by the DICOM message duct, wherein these identifiers reference the persisted data within the database 22. In certain embodiments, the registry 68 may be added to the output queue 48 of the DICOM message duct 10A to be provided to another DICOM data duct 10 or another portion of the DICOM data processing pipeline 12. Using this registry 68, the accumulator 46 ensures that all of the DICOM data of a related set of DICOM files (e.g., an association, series, or study) has been suitably processed and persisted, in accordance with the steps discussed above. For example, after processing the first ultrasound image of this example, in FIG. 5, the registry 68 of the accumulator 46 includes identifying information regarding the first ultrasound image (e.g., a message value that indicates the Study Instance UID and the SOP UID of the first image). Once the accumulator 46 populates the registry 68 with this information for the first ultrasound image, all of the representations 64 and dictionaries 40 of the DICOM data related to this first image are discarded from memory 18, which reduces the memory consumption of the duct 10A and/or pipeline 12.

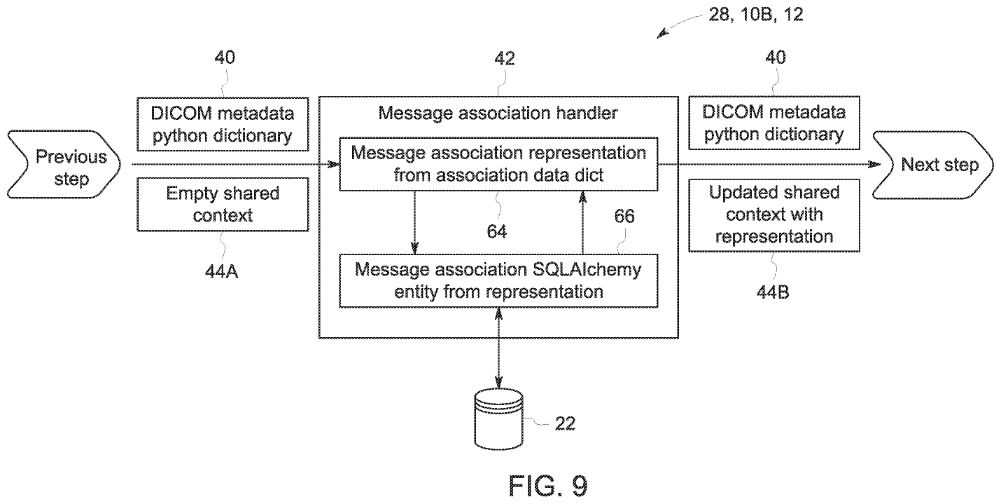

For the example embodiment of the DICOM message duct 10A, since the received DICOM data includes two ultrasound images, the second DICOM image of the association may be processed by the DICOM message duct using steps 1-3 discussed above. Since all of the representations 64 and dictionaries 40 of the DICOM data related to the first image were discarded at accumulation, only the information regarding the first image that is stored in the registry 68 of the accumulator 46 is available (e.g., in the shared context 44 of the DICOM message duct 10A) during the processing of the second image. As illustrated in FIG. 6, upon reaching the accumulator output stage 30 in the processing of the second image, the accumulator 46 updates the registry 68 to also include identifying information for the second image of the association, and then discards all of the representations 64 and dictionaries 40 related to the second image from the memory 18.

### DICOM Association Duct

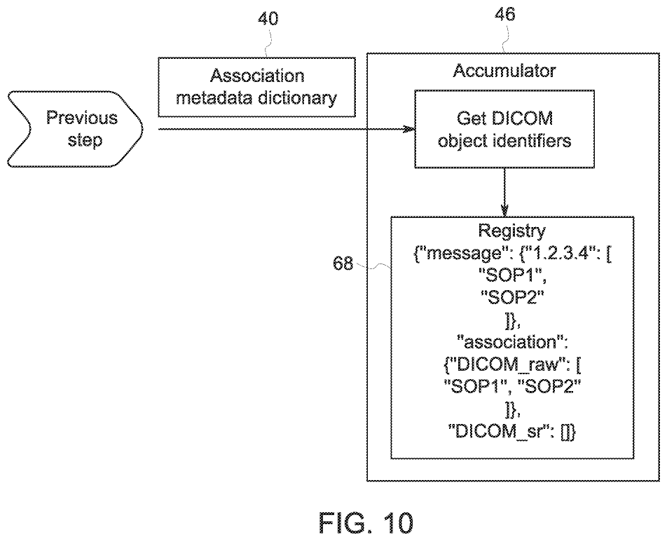

FIGS. 7-10 are diagrams illustrating an example embodiment of a DICOM association duct 10B, which is another example of an ingestion duct of a DICOM data processing pipeline 12. The purpose of the DICOM association duct 10B is generally to parse and store association data from DICOM association files available on the file system. A DICOM association file is a JSON file that stores metadata related to an association, wherein the association may be related to one or more images of a series or study. The DICOM association file is generated at the end of the association (e.g., after all images of the association have been collected) and may be suitably stored in a particular location in the file system.

For the example embodiment of the DICOM association duct 10B, FIG. 7 illustrates the acquisition stage 24. Each time a suitable DICOM association file 70 (e.g., a file having a .json extension) is written to a predetermined location in the file system of the storage 20, a watcher 32 of the duct 10B (e.g., a file system listener based on a Python watchdog library) adds the file URI into an input queue 36 of the duct 10B, which may be implemented as a Python queue in certain embodiments. Once the DICOM association file 70 has been added to the input queue 36, it becomes available for the parsing stage 26, as discussed below.

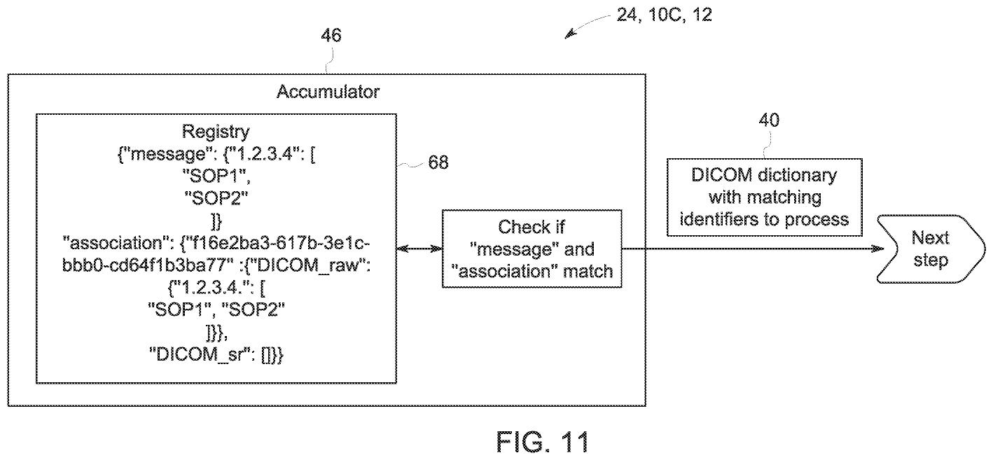

For the example embodiment of the DICOM association duct 10B, FIG. 8 illustrates the parsing stage 26, in which the DICOM association file 70 is parsed. Once the DICOM association file 70 is available in the input queue 36, it is opened and parsed by a JSON parser 38 (e.g., an association parser) to populate a DICOM dictionary 40 (e.g., a Python dictionary) with extracted DICOM data in memory 18. Unlike the DICOM parsing of the DICOM message duct 10A, the parsing of the DICOM association file 70 does not involve a specific configuration (e.g., a mapping file), since the structure can be fixed in the listener of the first step. Thus, the JSON parser 38 will only extract relevant data to be eventually persisted in a suitable database table, and will use this data to populate the DICOM dictionary 40 of the duct 10B. For this example, FIG. 8 illustrates example DICOM information that is extracted from the DICOM association file 70 during parsing to populate the DICOM dictionary 40.

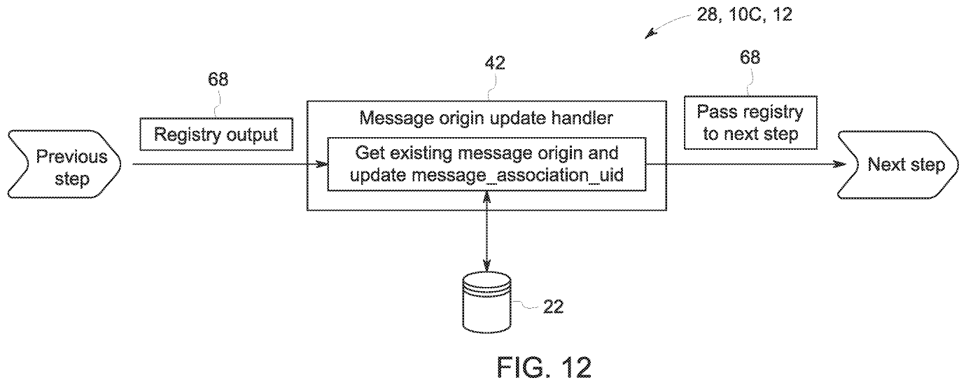

For the example embodiment of the DICOM association duct 10B, FIG. 9 illustrates the persistence output stage 28. For the illustrated example, once the DICOM dictionary 40 populated with the DICOM data from the DICOM association file 70 is available, it is provided to a suitable output handler 42 (e.g., a message association handler). Before the data from the DICOM dictionary 40 is written to the database 22, an intermediate object (e.g., a message association representation 64) is first created in memory 18. After creation, the representation 64 is persisted (e.g., stored within the database) using a suitable database object 66 (e.g., a SQLAlchemy entity). Additionally, the shared context 44A of the duct 10B is initially empty and is subsequently updated to yield the updated shared context 44B having suitable identifiers (e.g., database-generated identifiers, UIDs, SOP UIDs, key values) that reference the persisted data within the database 22. In certain embodiments, the relevant DICOM data extracted from the DICOM association file 70 during the parsing stage 26 may be persisted in a message_association table that is associated with the message_origin table of the database 22, which was populated by the DICOM message duct 10A, as discussed above, in this example.

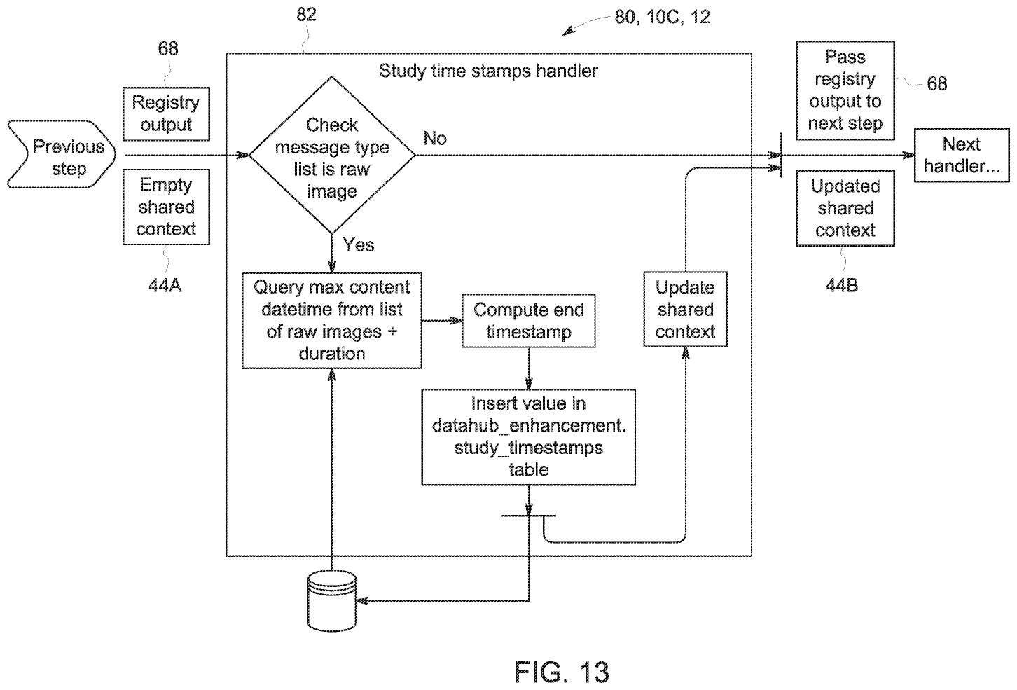

For the example embodiment of the DICOM association duct 10B, FIG. 10 illustrates an accumulator output stage 30. After the DICOM data from the DICOM association file 70 is stored in the database 22 in persistence output stage 28, the DICOM dictionary 40 is subsequently provided to the accumulator 46. In particular, for this example, the accumulator 46 is the same accumulator as described for the DICOM message duct 10A above (e.g., a shared accumulator), and may be part of a DICOM accumulation duct or an enhancement duct, in certain embodiments. The accumulator 46 generally receives the DICOM dictionary 40, accesses the shared context 44 of the DICOM association duct 10B, and updates the registry 68 to include suitable identifiers (e.g., identifying information, database-generated identifiers, UIDs, SOP UIDs, key values, associations) that reference the DICOM association data persisted within the database 22. For example, in certain embodiments, the registry 68 may be stored within an output queue 48 of the DICOM association duct 10B.

Using this registry 68, the accumulator 46 ensures that all of the DICOM association data of a related set of DICOM files (e.g., an association, series, or study) has been suitably processed and persisted, in accordance with the steps discussed above. For example, after processing the two ultrasound images in the DICOM message duct 10A (as discussed with respect to the example DICOM message duct 10A above) and processing the DICOM association file within the DICOM association duct 10B, in FIG. 10, the registry 68 of the accumulator 46 includes identifying information regarding: the DICOM message (e.g., a message value that indicates the Study Instance UID), the first ultrasound image (e.g., the SOP UID of the first image), the second ultrasound image (e.g., the SOP UID of the second image), and the DICOM association (e.g., a DICOM raw array that lists the SOP UIDs of the first and the second image of the association). Once the accumulator 46 populates the registry 68 with the desired data from the DICOM association file and the shared context 44, all of the representations 64 and dictionaries 40 of the DICOM data related to this association are discarded from the memory 18. Since the DICOM message duct 10A and the DICOM association duct 10B may operate independently and asynchronously prior to accumulation, in certain cases, the registry 68 of the accumulator 46 may not be complete after the association data is added, depending on whether the DICOM message duct 10A has completed processing and persisting the images of the association, as discussed with respect to the DICOM message duct 10A above.

### DICOM Enhancement Duct

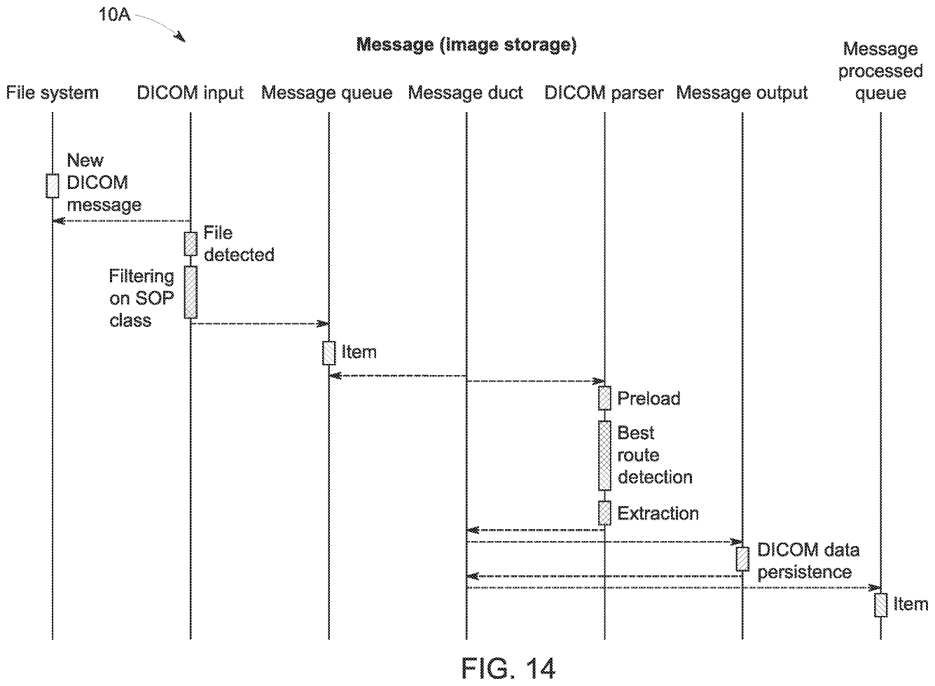

FIGS. 11-13 are diagrams illustrating an example embodiment of a DICOM enhancement duct 10C. The purpose of the DICOM enhancement duct is generally to start enhancement calculations, such as the end of a study.

For the example embodiment of the DICOM enhancement duct 10C, FIG. 11 illustrates the acquisition stage 24 (e.g., registry data acquisition) for the DICOM enhancement duct 10C. For this example, the shared accumulator 46 that received dictionaries 40 from the DICOM message duct 10A and the DICOM association duct 10B, provides the registry 68 as the input of the DICOM enhancement duct 10C. In other words, for the present example, the output queues 48 of the DICOM message duct 10A and the DICOM association duct 10B, which include the registry 68, serve as the input queue 36 of the DICOM enhancement duct 10C. As noted, in certain embodiments, the accumulator 46 may be part of an accumulation duct disposed between the ingestion ducts (e.g., the DICOM message duct 10A, the DICOM association duct 10B) and the DICOM enhancement duct 10C. For the embodiment illustrated in FIG. 11, the accumulator 46 includes a method that determines whether the “message” and “association” entries match (e.g., suitably correspond to one another) after each new addition to the registry. As such, once the accumulator 46 determines that the registry 68 is fully populated, and that the “message” and “association” entries suitably match, the registry 68 may be made available to the input queue 36 of the DICOM enhancement duct 10C. For the illustrated embodiment, the registry 68 is added to the input queue 36 of the duct 10C as a DICOM dictionary 40 or in another suitable form.

For the example embodiment of the DICOM enhancement duct 10C, once the registry 68 has been added to the input queue 36 of the duct 10C, it becomes available for the parsing stage 26 of the DICOM enhancement duct 10C. For the DICOM enhancement duct 10C, pass-through parsing may be used, wherein the information from the registry 68 (e.g., the DICOM identifiers and values) proceeds to the next step without modification.

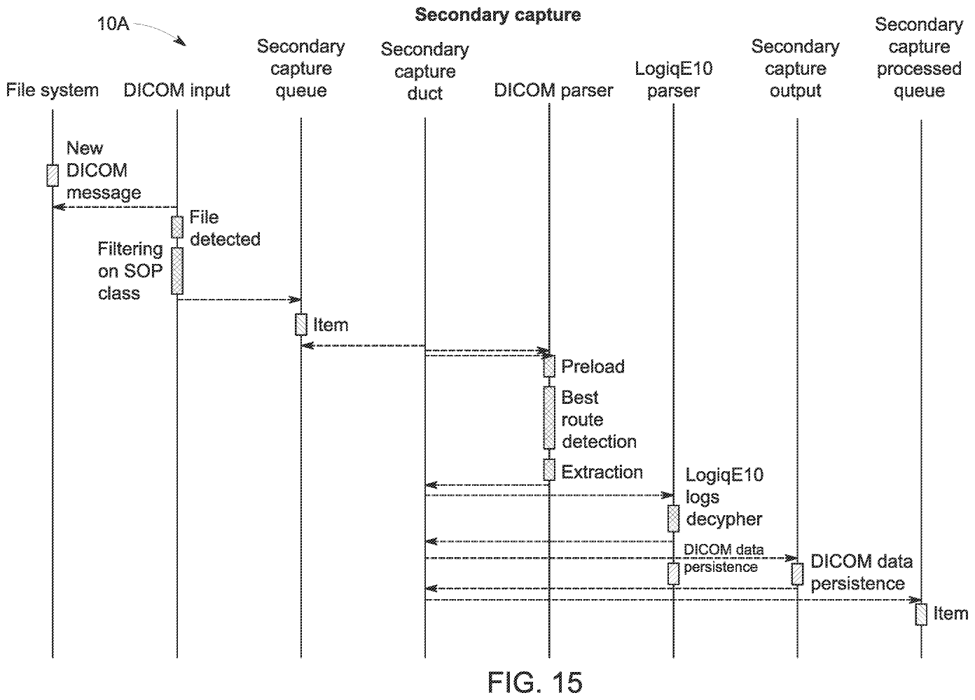

FIG. 12 illustrates the persistence output stage 28 of the example embodiment of the DICOM enhancement duct 10C. As noted above, the registry 68 may be provided to the persistence output stage 28 as a DICOM dictionary 40 or in another suitable form. In certain embodiments, one or more handlers 42 in the DICOM enhancement duct 10C store data in a distinct location (e.g., a different database schema) relative to the DICOM message duct 10A and the DICOM association duct 10B. However, in certain embodiments, at least one handler 42 of the DICOM enhancement duct 10C (e.g., a message origin update handler) is configured to update certain information stored in the database 22 by the DICOM message duct 10A and/or the DICOM association duct 10B. For this example, after the operation of the DICOM message duct 10A, a suitable database table (e.g., a message_origin table) stores a message UID, the corresponding file URI, and the SOP class of the DICOM message. As illustrated in FIG. 12, the handler 42 (e.g., a message origin update handler) of the duct 10C updates this table to include the UID of the association (e.g., updates a message_association_uid field in the message_origin table). It may be appreciated that this step enables the tracking of links between the DICOM association and the DICOM messages.

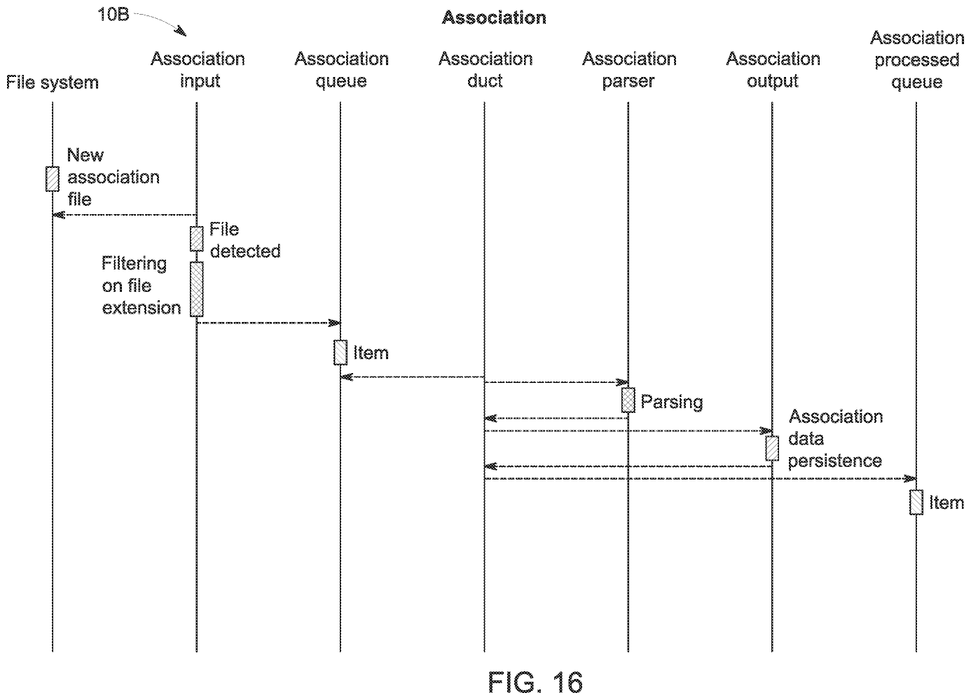

FIG. 13 illustrates an enhancement calculation stage 80 involving performing enhancement calculations in the example embodiment of the DICOM enhancement duct 10C. As noted above, in certain embodiments, the enhancement calculation stage 80 may be considered part of the persistence output stage 28 and/or the accumulator output stage 30 of the generic DICOM data duct 10, or may be considered an additional stage. For enhancement calculation stage 80, the DICOM enhancement duct 10C includes one or more handlers 82 configured to use the DICOM data that has been collected and processed by the ingestion ducts (e.g., the DICOM message duct 10A and the DICOM association duct 10B) to calculate metrics, such as the end of the exam, the age of the patient, and so forth. To avoid having all data objects being stored in memory 18, in certain embodiments, the handlers 82 may query any desired entities from the DICOM generic data model to perform the enhancement calculations. In particular, the handler 82 illustrated in FIG. 13 (e.g., study timestamps handler) determines “end of exam” timestamps based on the previously processed DICOM data, and stores these timestamps in a separate schema of the database 22 (e.g., in an enhancement_study_timestamp table). Additionally, while the shared context 44A of the duct 10C is initially empty, the shared context 44B is updated throughout operation of the handler 82. As such, the registry 68 and/or the updated shared context 44B of the duct 10C are made available to subsequent handlers of the duct 10C to perform additional enhancement calculations.

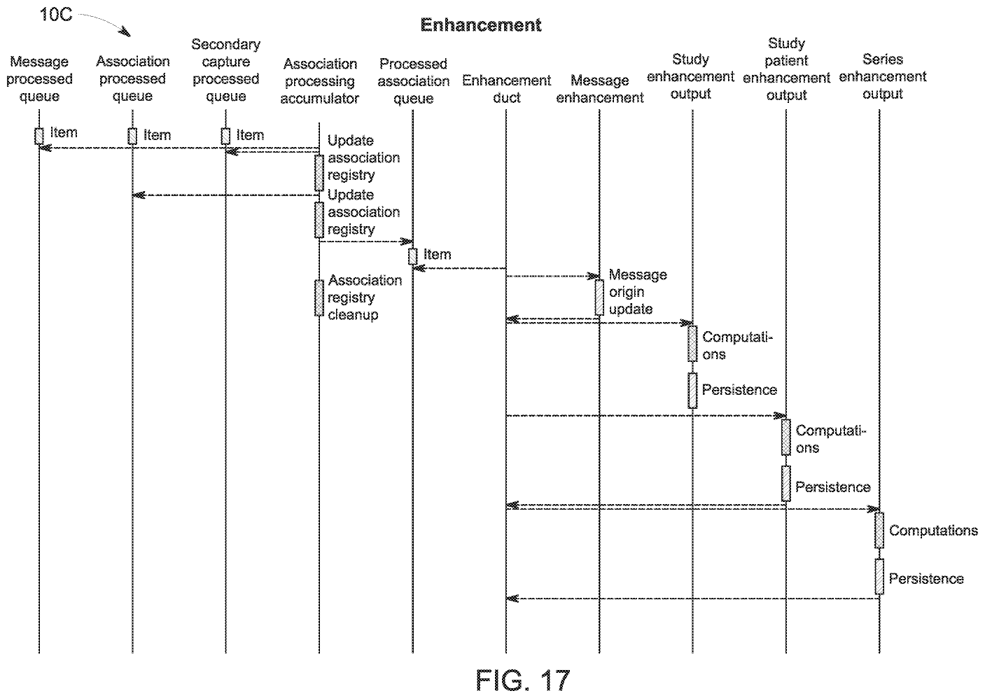

FIGS. 14-17 illustrate example communications between the components of various embodiments of DICOM data ducts 10, as well as other components of the computing system 14, for embodiments of the present approach. More specifically, FIG. 14 illustrates communication between system components for an embodiment of the DICOM message duct 10A. FIG. 15 illustrates communication between system components for an embodiment of the DICOM message duct 10A that performs secondary capture, wherein the duct includes a secondary parser that performs a secondary parsing step to decipher the secondary capture output. FIG. 16 illustrates communication between system components for an embodiment of the DICOM association duct 10B. FIG. 17 illustrates communication between system components for an embodiment of the DICOM enhancement duct 10C.

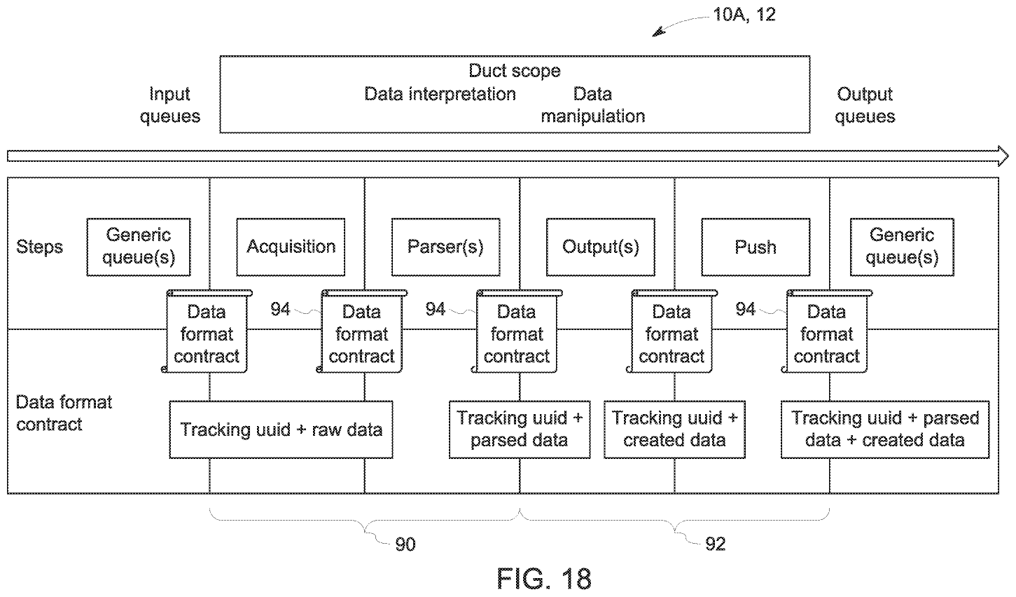

FIG. 18 is an alternative visualization of an embodiment of a DICOM message duct 10A as part of a DICOM data processing pipeline 12, as discussed above. In particular, in FIG. 18, the actions of the DICOM message duct 10A are broadly divided into a data interpretation stage 90 and a data manipulation stage 92. Additionally, the example DICOM message duct 10A of FIG. 18 emphasizes the enforcement of data format contracts 94 (also referred to herein simply as “contracts”) at each stage of the duct 10 (and the overall pipeline 12) to ensure that the data types and values of each input and each output correspond to the expected data types and values.

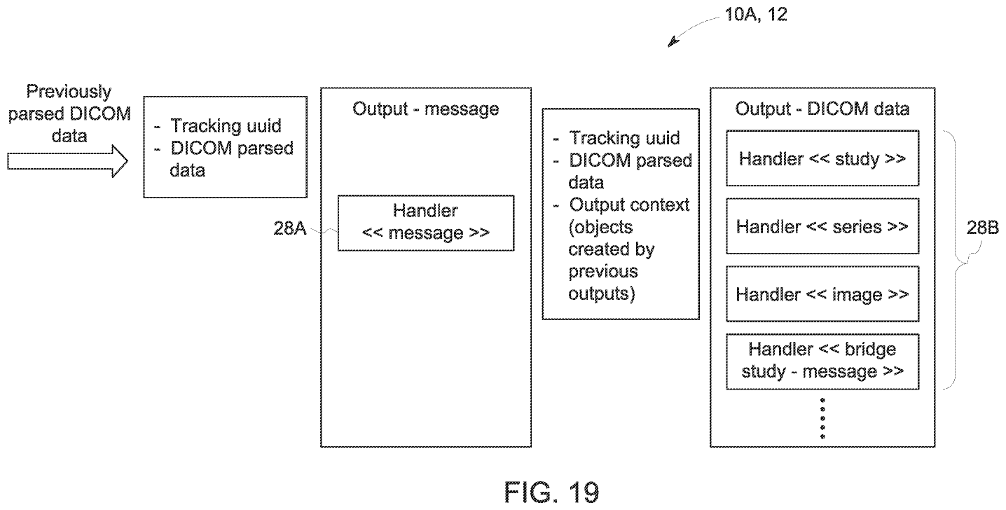

FIG. 19 is a diagram illustrating how multiple handlers 42 may cooperate to store data within the database 22 for an example embodiment of a DICOM message duct 10A. For the illustrated example, a primary handler 42A persists certain information related to the message, such as a tracking universal unique identifier (UUID). Subsequently, secondary handlers 42B of the DICOM message duct 10A persist certain study, series, image, and bridging information related to the message. In addition to the parsed DICOM data, these secondary handlers 42B also have access to the output context of the first handler via the shared context 44 of the DICOM message duct 10A, meaning that the handlers 42 can use these identifiers (e.g., the tracking UUID) to access and/or correlate DICOM message information before database output.

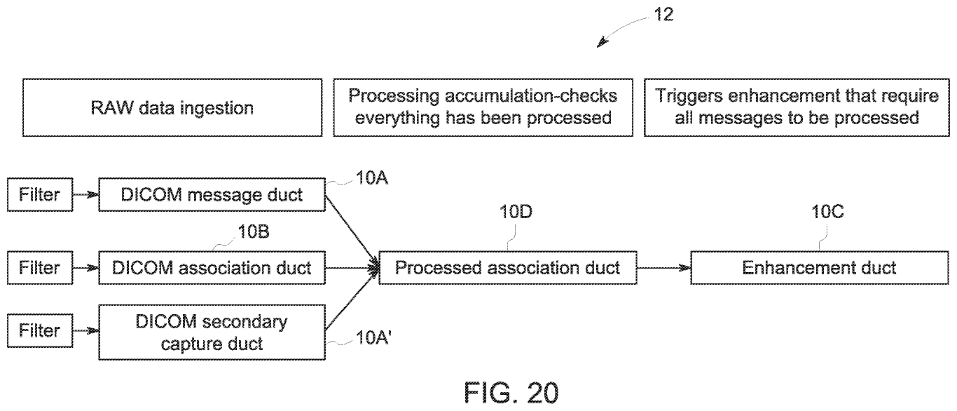

FIG. 20 is a diagram illustrating an example deployment of a DICOM data processing pipeline 12 that includes a plurality of DICOM data ducts 10, in accordance with embodiments of the present technique. As illustrated, the DICOM data processing pipeline 12 includes a number of raw data ingestion ducts, including a DICOM message duct 10A, a DICOM association duct 10B, and a DICOM secondary capture duct 10A′, as discussed above. Each of these ingestion ducts may be configured to operate independently and asynchronously from one another. The outputs of the ingestion ducts (e.g., dictionaries) are separately provided as inputs to a DICOM accumulation duct 10D (e.g., a processed association duct) that includes a shared accumulator. As discussed above, the shared accumulator 46 of the accumulation duct 10D ensures that all of the relevant data for a related set of DICOM files (e.g., an association, a study) has been processed by the ingestion ducts, and constructs the registry storing identifying information for the DICOM data stored within the database 22 by the various ingestion ducts. Once all of the relevant DICOM data has been ingested and accumulated, the populated and validated registry is provided as an input to a DICOM enhancement duct 10C, which performs additional calculations to generate new data based on the stored DICOM data. It may be appreciated that such a deployment offers advantages, such as limiting the number of processes and the corresponding computer resource usage, providing comprehensive duct scopes, and allowing parallelization during data ingestion.

Technical effects of the invention include improved processing of DICOM data. Present embodiments are directed to systems and methods for use in the processing of medical data. Compared to current streaming APIs, the disclosed DICOM data ducts enable data processing logic validation, data lineage in terms of end-to-end transformation, data processing audit logs, and error control management. The disclosed ducts provide a higher-level encapsulation of processing APIs and force contractual exchanges, which allows for logical validation and control of each processing step or each group of processing steps. By enforcing contractual exchanges, the ducts (as well as a larger data processing pipeline that includes these ducts) can be logically validated, both in terms of provided data and how this data is processed, which is important to businesses dealing with private information. More specifically, the disclosed ducts can ensure that various data processing pipelines are properly fed with the proper data and, if an error occurs, can track the error and easily assess its impact. It may be appreciated that this enables these ducts to provide automated data lineage generation. Additionally, the disclosed ducts support error policies, which allows for routing depending on when and where the error occurs in the duct or the data processing pipeline.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computing system, comprising:
at least one memory configured to store a database and instructions of a data processing pipeline for processing DICOM data related to a study; and
at least one processor configured to execute the stored instructions of the data processing pipeline to perform actions comprising:
ingesting, via a plurality of ingestion ducts of the data processing pipeline, a plurality of DICOM files of the study by: parsing each of the plurality of DICOM files to populate a corresponding plurality of dictionaries, storing the data of the plurality of dictionaries in the database, updating a shared context of the data processing pipeline with identifiers that reference the stored data of each of the plurality of dictionaries within the database, and providing the plurality of dictionaries as input to an accumulation duct of the data processing pipeline;
accumulating, via the accumulation duct, the plurality of dictionaries received from the plurality of ingestion ducts and the identifiers of the shared context to populate a registry, and in response to determining, based on the registry, that each of the plurality of DICOM files of the study has been ingested, providing the registry as input to an enhancement duct of the data processing pipeline; and
enhancing, via the enhancement duct, the stored data of the plurality of dictionaries within the database for the study, which is accessed within the database using the identifiers of the registry received from the accumulation duct.

2. The computing system of claim 1, wherein, to ingest the plurality of DICOM files, the at least one processor is configured to execute the stored instructions to perform actions comprising:
ingesting, via a DICOM message duct of the plurality of ingestion ducts, a DICOM message file of the study by: parsing the DICOM message file to populate a first dictionary with data from the DICOM message file, storing the data of the first dictionary in the database, updating the shared context with first identifiers that reference the stored data of the first dictionary within the database, and providing the first dictionary as input to the accumulation duct; and
ingesting, via a DICOM association duct of the plurality of ingestion ducts, a DICOM association file of the study by: parsing the DICOM association file to populate a second dictionary with data from the DICOM association file, storing the data of the second dictionary in the database, updating the shared context with second identifiers that reference the stored data of the second dictionary within the database, and providing the second dictionary as input to the accumulation duct.

3. The computing system of claim 2, wherein, to ingest the DICOM message file, the at least one processor is configured to execute the stored instructions of the DICOM message duct to perform actions comprising:
receiving, via an input queue of the DICOM message duct, a file uniform resource identifier (URI) or a file stream of a DICOM message file of the study;
parsing, via at least one parser of the DICOM message duct, the DICOM message file based on a mapping JavaScript Object Notation (JSON) file to populate the first dictionary of the plurality of dictionaries with content from one or more DICOM tags of the DICOM message file;
storing, via at least one database handler of the DICOM message duct, the first dictionary within the database, determining first identifiers that reference the stored data of the first dictionary within the database, and updating the shared context to include the first identifiers; and
adding the first dictionary to an output queue of the DICOM message duct that is associated with an input queue of the accumulation duct.

4. The computing system of claim 3, wherein, to store the first dictionary within the database, the at least one processor is configured to execute the stored instructions of the at least one database handler of the DICOM message duct to perform actions comprising:
creating an intermediate object in the at least one memory from the data of the first dictionary;
providing the intermediate object to a database object of the at least one database handler, wherein the database object is configured to store the intermediate object in the database, to receive the first identifiers from the database in response to storing the intermediate object, and to update the intermediate object to include the first identifiers;
updating the shared context based on the updated intermediate object; and
removing the intermediate object from the at least one memory.

5. The computing system of claim 2, wherein, to ingest the DICOM association file, the at least one processor is configured to execute the stored instructions of the DICOM association duct to perform actions comprising:
receiving, via an input queue of the DICOM association duct, a file uniform resource identifier (URI) or a file stream for the DICOM association file;
parsing, via a parser of the DICOM association duct, the DICOM association file to populate the second dictionary with content from one or more DICOM tags of the DICOM association file;
storing, via at least one database handler of the DICOM association duct, the second dictionary within the database, determining second identifiers that reference the stored data of the second dictionary within the database, and updating the shared context to include the second identifiers; and
adding the second dictionary to an output queue of the DICOM association duct that is associated with an input queue of the accumulation duct.

6. The computing system of claim 2, wherein the at least one processor is configured to execute the stored instructions of the data processing pipeline to perform actions comprising:
ingesting, via the DICOM message duct, a second DICOM message file of the study by: parsing the second DICOM message file to populate a third dictionary with data from the second DICOM message file, storing the data of the third dictionary in the database, updating the shared context with third identifiers that reference the stored data of the third dictionary within the database, and providing the third dictionary as input to the accumulation duct.

7. The computing system of claim 1, wherein, to accumulate the plurality of dictionaries and the identifiers, the at least one processor is configured to execute the stored instructions of the accumulation duct to perform actions comprising:
verifying that each of the plurality of DICOM files are part of the study based on the plurality of dictionaries, the identifiers of the shared context, or a combination thereof, before populating the registry.

8. The computing system of claim 1, wherein the at least one processor is configured to execute the stored instructions of the accumulation duct to perform actions comprising:
after populating the registry, removing the plurality of dictionaries from the at least one memory, wherein the registry is stored in the shared context of the data processing pipeline.

9. A computer-implemented method of operating a data processing pipeline, comprising:
ingesting, via a plurality of ingestion ducts of the data processing pipeline, a plurality of DICOM files of a study by: parsing each of the plurality of DICOM files to populate a corresponding plurality of dictionaries, storing the data of the plurality of dictionaries in a database, updating a shared context of the data processing pipeline with identifiers that reference the stored data of each of the plurality of dictionaries within the database, and providing the plurality of dictionaries as input to an accumulation duct of the data processing pipeline;
accumulating, via the accumulation duct, the plurality of dictionaries received from the plurality of ingestion ducts and the identifiers of the shared context to populate a registry, and in response to determining, based on the registry, that each of the plurality of DICOM files of the study has been ingested, providing the registry as input to an enhancement duct of the data processing pipeline; and
enhancing, via the enhancement duct, the stored data of the plurality of dictionaries within the database for the study, which is accessed within the database using the identifiers of the registry received from the accumulation duct.

10. The computer-implemented method of claim 9, wherein ingesting the plurality of DICOM files comprises:
ingesting, via a DICOM message duct of the plurality of ingestion ducts, a DICOM message file of the study by: parsing the DICOM message file to populate a first dictionary with data from the DICOM message file, storing the data of the first dictionary in the database, updating the shared context with first identifiers that reference the stored data of the first dictionary within the database, and providing the first dictionary as input to the accumulation duct; and
ingesting, via a DICOM association duct of the plurality of ingestion ducts, a DICOM association file of the study by: parsing the DICOM association file to populate a second dictionary with data from the DICOM association file, storing the data of the second dictionary in the database, updating the shared context with second identifiers that reference the stored data of the second dictionary within the database, and providing the second dictionary as input to the accumulation duct.

11. The computer-implemented method of claim 10, wherein ingesting the DICOM message file comprises:
receiving, via an input queue of the DICOM message duct, a file uniform resource identifier (URI) or a file stream of a DICOM message file of the study;
parsing, via at least one parser of the DICOM message duct, the DICOM message file based on a mapping JavaScript Object Notation (JSON) file to populate the first dictionary of the plurality of dictionaries with content from one or more DICOM tags of the DICOM message file;
storing, via at least one database handler of the DICOM message duct, the first dictionary within the database, determining first identifiers that reference the stored data of the first dictionary within the database, and updating the shared context to include the first identifiers; and
adding the first dictionary to an output queue of the DICOM message duct that is associated with an input queue of the accumulation duct.

12. The computer-implemented method of claim 11, wherein storing the first dictionary within the database comprises:
creating an intermediate object in memory from the data of the first dictionary;
providing the intermediate object to a database object of the at least one database handler, wherein the database object is configured to store the intermediate object in the database, to receive the first identifiers from the database in response to storing the intermediate object, and to update the intermediate object to include the first identifiers;
updating the shared context based on the updated intermediate object; and
removing the intermediate object from the memory.

13. The computer-implemented method of claim 10, wherein ingesting the DICOM association file comprises:
receiving, via an input queue of the DICOM association duct, a file uniform resource identifier (URI) or a file stream for the DICOM association file;
parsing, via a parser of the DICOM association duct, the DICOM association file to populate the second dictionary with content from one or more DICOM tags of the DICOM association file;
storing, via at least one database handler of the DICOM association duct, the second dictionary within the database, determining second identifiers that reference the stored data of the second dictionary within the database, and updating the shared context to include the second identifiers; and
adding the second dictionary to an output queue of the DICOM association duct that is associated with an input queue of the accumulation duct.

14. The computer-implemented method of claim 10, comprising:
ingesting, via the DICOM message duct, a second DICOM message file of the study by: parsing the second DICOM message file to populate a third dictionary with data from the second DICOM message file, storing the data of the third dictionary in the database, updating the shared context with third identifiers that reference the stored data of the third dictionary within the database, and providing the third dictionary as input to the accumulation duct.

15. The computer-implemented method of claim 9, wherein accumulating comprises:
verifying that each of the plurality of DICOM files are part of the study based on the plurality of dictionaries, the identifiers of the shared context, or a combination thereof, before populating the registry.

16. The computer-implemented method of claim 9, wherein enhancing comprises:
accessing, via the enhancement duct, the stored data of the plurality of dictionaries within the database using the identifiers of the registry received from the accumulation duct;
calculating additional data for the study based on the accessed data; and
storing the additional data for the study within the database.

17. A non-transitory, computer-readable medium storing instructions of a data processing pipeline executable by a processor of a computing system, the instructions comprising instructions to:
ingest, via a plurality of ingestion ducts of the data processing pipeline, a plurality of DICOM files of a study by: parsing each of the plurality of DICOM files to populate a corresponding plurality of dictionaries, storing the data of the plurality of dictionaries in a database, updating a shared context of the data processing pipeline with identifiers that reference the stored data of each of the plurality of dictionaries within the database, and providing the plurality of dictionaries as input to an accumulation duct of the data processing pipeline;
accumulate, via the accumulation duct, the plurality of dictionaries received from the plurality of ingestion ducts and the identifiers of the shared context to populate a registry, and in response to determining, based on the registry, that each of the plurality of DICOM files of the study has been ingested, providing the registry as input to an enhancement duct of the data processing pipeline; and
enhance, via the enhancement duct, the stored data of the plurality of dictionaries within the database for the study, which is accessed within the database using the identifiers of the registry received from the accumulation duct.

18. The non-transitory, computer-readable medium of claim 17, wherein the instructions to ingest the plurality of DICOM files comprise instructions to:
ingest, via a DICOM message duct of the plurality of ingestion ducts, a DICOM message file of the study by: parsing the DICOM message file to populate a first dictionary with data from the DICOM message file, storing the data of the first dictionary in the database, updating the shared context with first identifiers that reference the stored data of the first dictionary within the database, and providing the first dictionary as input to the accumulation duct; and
ingest, via a DICOM association duct of the plurality of ingestion ducts, a DICOM association file of the study by: parsing the DICOM association file to populate a second dictionary with data from the DICOM association file, storing the data of the second dictionary in the database, updating the shared context with second identifiers that reference the stored data of the second dictionary within the database, and providing the second dictionary as input to the accumulation duct.

19. The non-transitory, computer-readable medium of claim 18, wherein the instructions to ingest the DICOM message file comprise instructions to:
receive, via an input queue of the DICOM message duct, a file uniform resource identifier (URI) or a file stream of a DICOM message file of the study;
parse, via at least one parser of the DICOM message duct, the DICOM message file based on a mapping JavaScript Object Notation (JSON) file to populate the first dictionary of the plurality of dictionaries with content from one or more DICOM tags of the DICOM message file;
store, via at least one database handler of the DICOM message duct, the first dictionary within the database, determine first identifiers that reference the stored data of the first dictionary within the database, and update the shared context to include the first identifiers; and
add the first dictionary to an output queue of the DICOM message duct that is associated with an input queue of the accumulation duct.

20. The non-transitory, computer-readable medium of claim 19, wherein the instructions to store the first dictionary within the database comprise instructions to:
create an intermediate object in a memory from the data of the first dictionary;
provide the intermediate object to a database object of the at least one database handler, wherein the database object is configured to store the intermediate object in the database, to receive the first identifiers from the database in response to storing the intermediate object, and to update the intermediate object to include the first identifiers;
update the shared context based on the updated intermediate object; and
remove the intermediate object from the memory.
